SEQUENCE LISTING (3 sequences)

SEQ ID NO: 1 - RNA, 36 nt, Artificial sequence
  crRNA antiTRAC
agagucucucagcugguacaguuuuagagcuaugcu

SEQ ID NO: 2 - RNA, 67 nt, Artificial sequence
  tracRNA
agcauagcaaguuaaaauaaggcuaguccguuaucaacuugaaaaguggcaccgagucggugcuuuu

SEQ ID NO: 3 - RNA, 36 nt, Artificial sequence
  crRNA antiB2M
ggccacggagcgagacaucuguuuuagagcuaugcu

(19) United States
(12) Patent Application Publication
(10) Pub. No.: US 2022/0380726 A1
(43) Pub. Date: Dec. 1, 2022

(54) UNIVERSAL CAR-T CELL AND PREPARATION AND USE THEREOF

(71) Applicant: Cellular Biomedicine Group HK Limited, Hong Kong (CN)

(72) Inventors: Yun JI, Shanghai (CN); Wei-chin YANG, Shanghai (CN); Huijuan CUI, Shanghai (CN); Xiaobing LUO, Rockville, MD (US); Jiaqi HUANG, Rockville, MD (US); Shigui ZHU, Rockville, MD (US); Xin YAO, Rockville, MD (US); Yihong YAO, Rockville, MD (US)

(21) Appl. No.: 17/631,006

(22) PCT Filed: Aug. 3, 2020
(86) PCT No.: PCT/CN2020/106635

§ 371 Date: Aug. 9, 2022

(30) Foreign Application Priority Data
Aug. 1, 2019 (CN) 201910708725.9

(51) Int. Cl. C12N 5/0783; A61K 35/17

(52) U.S. Cl. CPC C12N 5/0636; A61K 35/17; C12N 2501/2315; C12N 2501/2307; C12N 2501/2321

(57) ABSTRACT

Provided is a non-naturally occurring T population, of which the proportion of T memory stem cells satisfies C1≥50%. The T cell population comprises a tumor antigen-targeting universal CAR-T cell. Further provided is a method for preparing the CAR-T cell. The CAR-T cell is higher in amplification rate and better in phenotype, IFN-gamma releasing capacity and target cell killing capability.

### TECHNICAL FIELD The present invention relates to the field of biomedicine, and more particularly to universal CAR-T cells and preparation and use thereof

### BACKGROUND TECHNOLOGY

Immunotherapy, especially adoptive T-cell therapy, has shown strong efficacy and good prospect in clinical trials for the treatment of malignant tumors of the hematological system. T cells can be genetically modified to express the chimeric antigen receptor (CAR), which includes an antigen recognition moiety and a T cell activation domain. CARs use the antigen-binding properties of monoclonal antibodies to redirect T cell specificity and reactivity and direct at the targets in an MHC-unrestricted manner. This MHC-unrestricted antigen recognition enables CAR-expressing T cells to recognize antigens without antigen processing, thus avoiding a major mechanism of tumor escape. In addition, CARs do not dimerize with the α chain and β chain of endogenous TCR.

Universal CAR-T cells are an important alternative resource for autologous T cells in the T-cell immunotherapy, wherein endogenous TRAC and B2M genes of allogeneic T cells are knocked out to prevent the graft-versus-host disease and host immune rejection, respectively. These genetically edited cells from allogeneic donors offer cellular immunotherapy opportunities for patients who cannot provide enough autologous T cells, e.g., infants or patients who have received multiple cycles of chemotherapy.

At present, there is no universal CAR-T cell product approved for marketing at home and abroad. There are still many defects in the research of universal CAR-T cells in this field, and there is an urgent need to develop new universal CAR-T cell products with good killing effects and high safety.

### SUMMARY OF THE INVENTION

The purpose of the present invention is to provide Tscm-enriched universal CAR-T cells and preparation and use thereof.

In a first aspect, the present invention provides a non-naturally occurring T cell population, wherein the proportion of T memory stem cells (stem cell-like memory T cells, Tscm) in the T cell population, or C1≥50%, based on the total count of T cells in the T cell population.

In another preferred example, the C1≥60%, preferably C1≥70%, more preferably C1≥75%.

In another preferred example, the C1 is 50-85%, preferably 60-80%.

In another preferred example, the T cells include CAR-T cells.

In another preferred example, the CAR-T cells include autologous or allogeneic CAR-T cells.

In another preferred example, the CAR-T cells include universal CAR-T cells.

In another preferred example, in the CAR-T cells, an immune-related gene selected from the following group is knocked out or down-regulated: TRAC gene, B2M gene, or a combination thereof.

In another preferred example, when the T memory stem cells (Tscm) are CAR-T cells, the proportion Cl of the T memory stem cells (Tscm) is ≥50%, based on the total number of CAR-T cells in the T cell population.

In another preferred example, the C1≥60%, preferably C1≥70%, more preferably C1≥75%.

In another preferred example, the C1 is 50-85%, preferably 60-80%.

In another preferred example, the T memory stem cells have a CCR7⁺ phenotype.

In another preferred example, in the T cell population, the content of T cells C3≥30%, preferably ≥50%, more preferably ≥70%, more preferably ≥80%, ≥90%, or ≥95%, based on the total number of all cells in the cell population.

In another preferred example, the C3 is 30-100%, more preferably 70-99.9%, more preferably 80-99%.

In another preferred example, the T memory stem cells include CCR 7⁺CD45RA⁺T cells.

In another preferred example, in the T cell population, the proportion of those expressing the biomarker CCR7, or C2 is ≥50%, preferably ≥60%, more preferably ≥70%.

In another preferred example, the activity intensity Q1 of the CAR-T cells (take the mean fluorescence intensity MFI as an example) ≥500, preferably ≥1000, more preferably ≥3000, more preferably ≥4000 (e.g., 1000-5000).

In another preferred example, the T cell population is or is not amplified in vitro.

In another preferred example, the in vitro amplification is performed under conditions suitable for culture, and cell amplification is conducted in a culture system in the presence of IL15, IL7 and IL21 (commonly referred to as Tscm amplifying factors).

In another preferred example, in the culture system, the concentrations of the Tscm amplifying factors are:

The concentration of IL15 is 1-200 ng/ml, preferably, 3-100 ng/ml, more preferably 5-20 ng/ml;

The concentration of IL7 is 0.5-50 ng/ml, preferably, 1-20 ng/ml, more preferably 3-10 ng/ml; and/or

The concentration of IL21 is 1-100 ng/ml, preferably, 3-50 ng/ml, more preferably 5-20 ng/ml.

In another preferred example, the culture system includes a serum culture system and a serum-free culture system. In another preferred example, the cell amplification duration is 0.1-30 days, preferably 0.5-25 days, more preferably 1-20 days, most preferably 2-15 days or 3-15 days or 5-15 days.

In another preferred example, the CAR-T cells are universal CAR-T cells targeting tumor antigens.

In another preferred example, the tumor antigens are selected from the group of TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, mesothelin, IL-11Ra, PSCA, PRSS21, VEGFR2, LewisY, CD24, PDGFR-β, SSEA-4 , CD20, folate receptor α, ERBB2 (Her2/neu), MUC1, EGFR, NCAM, Prostase, PAP, ELF2M, Ephrin B2, IGF-I receptor, CAIX, LMP2, gp100, bcr-abl, tyrosine acidase, EphA2, fucosyl GM1, sLe, GM3, TGSS, HMWMAA, o-acetyl-GD2, folate receptor β, TEM1/CD248, TEM7R, CLDN6, GPRCSD, CXORF61, CD97, CD179a, ALK, polysialic Acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoint, ML-IAP, ERG (TMPRSS2ETS fusion gene), NA17, PAX3, androgen receptor, cyclin B1, MYCN, RhoC, TRP-2, CYP1B1, BORIS, SART3, PAXS, OY-TES1, LCK, AKAP-4, SSX2, RAGE-1, human telomerase reverse transcriptase, RU1, RU2, intestinal carboxylesterase, mut hsp70-2, CD79a, CD79b, CD72, LAIR1, FCAR, LILRA2, CD300LF, CLEC12A, BST2, EMR2, LY75, GPC3, FCRLS, IGLL1, or a combination thereof.

In another preferred example, the CAR-T cell expresses a chimeric antigen receptor, and the structure of the chimeric antigen receptor is shown in the following formula I:

L—scFv—H—TM—C—CD3ζ (I)

In the formula,
each “—” is independently a linking peptide or peptide bond;
L is an optional signal peptide sequence;
scFv is an antibody single-chain variable region sequence targeting a tumor antigen;
H is an optional hinge region;

TM is the transmembrane domain;
C is or is not a costimulatory signal molecule;
CD3ζ is a cytoplasmic signaling sequence derived from CD3ζ.
In another preferred example, the CAR-T cells are universal CAR-T cells targeting BCMA.
In another preferred example, the scFv targets BCMA.

In another preferred example, the L is a signal peptide of a protein selected from the group of CD8, GM-CSF, CD4, CD 137, or a combination thereof.

In another preferred example, the H is a hinge region of a protein selected from the group of CD8, CD28, CD137, or a combination thereof.

In another preferred example, the TM is a transmembrane region of a protein selected from the group of CD28, CD3 epsilon, CD45, CD4, CDS, CD8, CD9, CD16, CD22, CD33, CD37, CD64, CD80, CD86, CD134, CD137, CD154, or a combination thereof.

In another preferred example, the C is a costimulatory signal molecule selected from the following group of proteins: OX40, CD2, CD7, CD27, CD28, CD30, CD40, CD70, CD134, 4-1BB (CD137), PD1, Dap10, CDS, ICAM-1, LFA-1 (CD11a/CD18), ICOS (CD278), NKG2D, GITR, TLR2, or a combination thereof.

In another preferred example, the C includes a costimulatory signal molecule derived from 4-1BB, and/or a costimulatory signal molecule derived from CD28.

In another preferred example, the T cells are from human or non-human mammals.

In another preferred example, the T cell population is prepared by the method described in the third aspect of the present invention.

In the second aspect, the present invention provides a cell preparation, the cell preparation comprising the non-naturally occurring T cell population according to the first aspect of the present invention and a pharmaceutically acceptable carrier, diluent or excipient.

In a third aspect, the present invention provides a method for preparing CAR-T cells, comprising the steps of:
(a) Providing an isolated T cell;
(b) Amplifying and culturing the isolated T cells in the presence of IL15, IL7 and IL21, thereby obtaining cultured T cells; and
(c) Transforming the cultured T cells to prepare CAR-T cells.

In another preferred example, in step (a), the isolated T cells are selected from the group of naive T cells (Tn cells), Tn cell-rich cell populations, or total T cells, or a combination thereof.

In another preferred example, the isolated T cells are naive T cells (Tn cells) or a Tn cell-rich cell population.

In another preferred example, the T cells include autologous or allogeneic T cells.

In another preferred example, in step (b), when the isolated T cells are amplified and cultured, the concentration of IL15 in the culture system is 1-200 ng/ml, preferably 3-100 ng/ml, more preferably, 5-20 ng/ml; the concentration of IL7 is 0.5-50 ng/ml, preferably, 1-20 ng/ml, more preferably 3-10 ng/ml; and the concentration of IL21 is 1-100 ng/ml, preferably, 3-50 ng/ml, more preferably 5-20 ng/ml.

In another preferred example, the method is used to prepare universal CAR-T cells.

In another preferred example, the CAR-T cells target the tumor antigen BCMA.

In another preferred example, in step (a), the T cells are isolated from human peripheral blood, preferably, mononuclear cells from peripheral blood.

In another preferred example, the isolated T cells include total T cells and naive T cells (Tn cells).

In another preferred example, the naive T cells are CCR7⁺CD45RA⁺T cells.

In another preferred example, in step (b), the proliferation and culture is performed in an AIM-V medium.

In another preferred example, in step (b), the culture system further comprises FBS, Glutamax, Pen/Strip, or a combination thereof; preferably, the concentration of the FBS is 5-20%.

In another preferred example, in step (c), the transformation includes T cell activation, T cell nuclear transfection, and lentiviral transduction.

In another preferred example, in step (c), the transformation comprises the following steps:
(1) Using CD3/CD28 magnetic beads to activate the cultured T cells, and removing the magnetic beads to obtain the activated T cells;
(2) Using the CRISPR-Cas9 system to perform nucleofection of the activated T cells to knock out the TRAC gene and/or B2M gene of the T cells, thereby obtaining nucleofected T cells; and
(3) Using a viral vector containing a CAR expression kit to transduce the nucleofected T cells to prepare CAR-T cells.

In a fourth aspect, the present invention provides a method for preparing CAR-T cells, comprising the steps of:
(i) Providing an isolated Tn cell,
(ii) Optionally, proliferating and culturing the isolated T cell in the presence of IL15, IL7 and IL21 to obtain cultured T cells; and
(iii) Transforming the T cells to prepare CAR-T cells.

In the fifth aspect, the present invention provides a use of the T cell population (especially CAR-T cells) according to the first aspect of the present invention, or the cell preparation according to the second aspect of the present invention, for preparing a drug for preventing and/or treating cancers or tumors.

In another preferred example, the tumors are selected from the group of hematological tumors, solid tumors, or a combination thereof.

In another preferred example, the hematological tumors are selected from the group of acute myeloid leukemia (AML), multiple myeloma (MM), chronic lymphocytic leukemia (CLL), acute lymphoblastic leukemia (ALL), diffuse large B-cell lymphoma (DLBCL), or a combination thereof.

In another preferred example, the solid tumors are selected from the group of gastric cancer, gastric cancer peritoneal metastasis, liver cancer, leukemia, kidney tumor, lung cancer, small intestine cancer, bone cancer, prostate cancer, colorectal cancer, breast cancer, colorectal cancer, cervical cancer, ovarian cancer, lymphoma, nasopharyngeal cancer, adrenal tumor, bladder tumor, non-small cell lung cancer (NSCLC), glioma, endometrial cancer, testicular cancer, colorectal cancer, urinary tract cancer, thyroid cancer, or a combination thereof.

In another preferred example, the solid tumors are selected from the group of ovarian cancer, mesothelioma, lung cancer, pancreatic cancer, breast cancer, liver cancer, endometrial cancer, or a combination thereof.

In the sixth aspect, the present invention provides a use of the T cell population according to the first aspect of the present invention, or the cell preparation according to the second aspect of the present invention, for preventing and/or treating cancers or tumors.

In the seventh aspect, the present invention provides a method for treating diseases, comprising: administering an appropriate amount of the T cell population according to the first aspect of the present invention or the cell preparation according to the second aspect of the present invention to a subject requiring the treatment.

In another preferred example, the diseases are cancers or tumors. It should be understood that, without departing from the spirit of the present invention, the above-mentioned technical features of the present invention and the technical features specifically described below (e.g., examples) can be combined, thereby forming new or preferred technical solutions. For simplicity, they are not further described herein.

### DESCRIPTION OF THE DRAWINGS

FIG. 1 shows TRAC and B2M gene knockout of the universal T cells and purification rates detected by flow cytometry staining.

FIG. 2 shows BCMA CAR transduction rates of the universal T cells detected by flow cytometry staining.

FIG. 3 shows ELISA assay of the IFN-γ release after co-culture of the universal CAR-T cells and BCMA-expressing target cells.

FIG. 4 shows the phenotype and purity of the naive T cells isolated from PMMCs detected by flow cytometry.

FIG. 5 shows the amplification rates of three BCMA CAR-T cells.

FIG. 6 shows the proportions of various T cell subtypes of the three BCMA CAR-T cells and their CCR7expression levels.

FIG. 7 shows the CAR transfection rates of three BCMA CAR-T cells detected by flow cytometry.

FIG. 8 shows the release of IFN-γ after co-culture of three BCMA CAR-T cells and BCMA-expressing target cells.

FIG. 9 shows the killing rates of the three BCMA CAR-T cells against BCMA-expressing target cells.

FIG. 10 shows the enrichment and cell growth of the Tscm cells in T cells cultured under different conditions.

FIG. 11 shows the BCMA CAR transduction rates of T cells cultured under different conditions.

FIG. 12 shows the release of IFN-γ following BCMA CAR transduction of T cells cultured under different conditions.

FIG. 13 shows the enrichment of CCR7⁺CD45RA⁺Tscm cells of the three BCMA CAR-T cells.

The symbols used in the accompanying drawings of the present invention have the following meanings:

Mock: T cells without Cas9 RNP nucleofection; mock+CAR: T cells without Cas9 RNP nucleofection but transduced with BCMA CAR; CAR: T cells transduced with BCMA CAR; Double KO: T cells with TRAC and B2M double knockout after Cas9 RNP nucleofection; Double KO+CAR: T cells of the above-mentioned Double KO cells after transduction with BCMA CAR; BCMA CAR: traditional autologous CAR-T cells after transduction with BCMA CAR; 1° U CAR: first generation universal T cells transduced with BCMA CAR; 2° U CAR: second generation universal T cells transduced with BCMA CAR.

### SPECIFIC EMBODIMENTS

Through extensive and in-depth research, the inventors unexpectedly discovered for the first time that Tscm-enriched universal BCMA CAR-T cells prepared by culturing T cells under conditions conducive to T memory stem cell (Tscm) amplification showed better amplification rate, phenotype, IFN-γ release capacity and target cell killing capacity than the first generation thereof. Based on the enriched Tscm-enriched T cells of the present invention (especially the second-generation universal CAR-T cells), it was possible to prepare highly active universal CAR-T cells for different targets, and hence the present invention.

Specifically, to further improve the efficacy of universal BCMA CAR-T cells, a new second generation of Tscm-enriched universal CAR-T cells was prepared by culturing T cells under conditions conducive to amplification of T memory stem cells (Tscm), and such CAR-T cells were much better than the first-generation universal CAR-T cells in amplification, phenotype, and killing of target cells, indicating that the second-generation universal CAR-T cells are more optimized universal CAR-T cell products.

Typically, nucleofection can be used to deliver Cas9 RNP complexes to T cells to simultaneously knock out endogenous TRAC and B2M genes, and after purification, high-purity universal T cells with TRAC and B2M double knockouts can be prepared. BCMA CAR expression was introduced into the obtained T cells by using lentiviral transduction to generate first-generation universal BCAM CAR-T cells. These CAR-T cells could effectively kill target cells with an activity similar to that of autologous CAR-T cells without gene editing.

Terms Used

For a better understanding of the present disclosure, some terms are firstly defined. As used in this application, unless expressly stated otherwise herein, each of the following terms shall have the meaning given below. Additional definitions are also given throughout the application.

The term “approximately” may refer to a value or composition within an acceptable error range of a particular value or composition as determined by those ordinary skill in the art, which will depend in part on how the value or composition is measured or determined.

The term “administration” refers to the physical introduction of a product of the invention into a subject using any of a variety of methods and delivery systems known to those skilled in the art, including intravenously, intramuscularly, subcutaneously, intraperitoneally, spinally or other routes of parenteral administration, such as by injection or infusion.

The term “antibody” (Ab) shall include, but is not limited to, an immunoglobulin that specifically binds an antigen and comprises at least two heavy (H) chains and two light (L) chains interconnected by disulfide bonds, or its antigen-binding part. Each H chain comprises a heavy chain variable region (abbreviated as VH herein) and a heavy chain constant region. The heavy chain constant region contains three constant domains, CH1, CH2 and CH3. Each light chain comprises a light chain variable region (abbreviated as VH herein) and a light chain constant region. The light chain constant region contains one constant domain, CL. The VH and VL regions can be further subdivided into hypervariable regions called complementarity determining regions (CDRs), which contain dispersed, more conserved regions called framework regions (FRs). Each VH and VL contains three CDRs and four FRs, arranged from amino-terminus to carboxy-terminus in the following order: FR1, CDR1, FR2, CDR2, FR3, CDR3, FR4. The variable regions of the heavy chain and light chain contain binding domains that interact with an antigen.

It should be understood that the names of amino acids used herein are identified by their international single English letters, and the corresponding three English letter abbreviations of the amino acid names are: Ala(A), Arg(R), Asn (N), Asp(D), Cys (C), Gln(Q), Glu(E), Gly (G), His(H), Ile(I), Leu(L), Lys(K), Met(M), Phe (F), Pro (P), Ser (S), Thr (T), Trp (W), Tyr (Y), Val (V).

Universal CAR-T Cell

Universal T cells are an important alternative resource for autologous T cells in T cell immunotherapy, in which the endogenous TRAC and B2M genes of allogeneic T cells are knocked out to prevent graft-versus-host disease and host immune rejection, respectively. These genetically edited cells from allogeneic donors provide cellular immunotherapy opportunities for patients who do not have enough autologous T cells, e.g., infants or patients who have received multiple cycles of chemotherapy. These cells can be further modified by lentiviral vectors encoding CAR or TCR to generate allogeneic CAR/TCR T cells.

In the present invention, the Cas9 RNP complex was delivered into T cells by using nucleofection to simultaneously knock out endogenous TRAC and B2M genes. BCMA CAR expression was introduced by lentiviral transduction to generate readily available BCAM CAR-T cells. These CAR-T cells could effectively kill target cells with an activity similar to that of autologous BCMA CAR-T cells without gene editing.

To further enhance the viability of universal CAR-T cells, gene editing started from allogeneic naive T cells (Tn), and special culture conditions that favor the amplification of stem cell-like memory T cells (Tscm) are used to produce second-generation universal BCMA CAR-T cells with enriched Tscm. Such CAR-T cells are much better than traditional first-generation universal CAR-T cells in terms of amplification, phenotype, IFN-γ release capacity, and killing of target cells.

Tn Cell

Tn cells, or naive T cells (Tn), also known as unsensitized T cells, which develop and are mature in the thymus and migrate to peripheral lymphoid tissues, where they are in a relatively quiescent state before exposure to antigenic stimulation.

Tscm Cell

Tscm cell represents T memory stem cell, also known as stem cell memory T cell (Tscm)

BCMA

BCMA is a B cell maturation antigen, also known as CD269 or TNFRSF17, a member of the tumor necrosis factor receptor superfamily, and its ligands are the B cell activating factor (BAFF) and the proliferation-inducing ligand (APRIL).

Binding of BCMA to BAFF and APRIL triggers NF-kB activation, inducing upregulation of anti-apoptotic Bcl-2 members such as Bcl-xL or Bcl-2 and Mcl-1. The interaction between BCMA and its ligands regulates humoral immunity from different aspects as well as the growth and differentiation of B cells to maintain the stable balance of the human body environment.

BCMA expression is restricted to B cell lineages, expressed on plasmablasts, plasma cells, and some of mature B cells, increases upon differentiation of terminal B cells, and it is not expressed on most B cells, such as naive B cells, memory B cells, and B cells germinal centers and other organs. It was reported that the expression of BCMA was important for long-lived, sessile plasma cells in the bone marrow. Therefore, BCMA-deficient mice had reduced plasma cells in the bone marrow while their plasma cell levels in the spleen were unaffected. Mature B cells differentiated normally into plasma cells in BCMA knockout mice. The BCMA knockout mice looked normal, seemed healthy, and had normal numbers of B cells, but plasma cells could not have a long-term survival.

BCMA is also highly expressed in malignant plasma cells, such as multiple myeloma and plasma cell leukemia, and BCMA has also been detected in HRS cells of patients with Hodgkin lymphoma. In the United States, hematological malignancies account for about 10% of all malignant tumors, and myeloma accounts for 15% of all hematological malignancies. It has been reported in the literature that the expression of BCMA is associated with disease progression of multiple myeloma. The BCMA gene is highly expressed in myeloma samples, but its expression is very low in chronic lymphocytic leukemia, acute lymphocytic leukemia, and acute T-cell lymphocytic leukemia. B-cell lymphomas increased markedly in mouse models overexpressing the BCMA ligands BAFF and APRIL. Ligands binding to BCMA have been shown to regulate the growth and survival of BCMA-expressing multiple myeloma cells. Binding of BCMA to BAFF and APRIL allows the survival of malignant plasma cells, so depletion of BCMA-expressing tumor cells and disruption of the interaction between BCMA ligands and receptors may improve the treatment outcomes of multiple myeloma or other BCMA-positive B lineage malign lymphomas.

Chimeric Antigen Receptor (CAR)

The CAR of the present invention may comprise any type of antigen binding domain that targets tumor antigens.

The chimeric antigen receptor (CAR) of the present invention includes an extracellular domain, a transmembrane domain, and an intracellular domain. The extracellular domain includes a target-specific binding element (also known as antigen-binding domain). The intracellular domain includes a costimulatory signaling region and a ζ chain portion. The costimulatory signaling region refers to a portion of an intracellular domain that includes a costimulatory molecule. Rather than antigen receptors or their ligands, costimulatory molecules are cell surface molecules that are required for an efficient lymphocyte response to an antigen.

A linker can be incorporated between the extracellular domain and the transmembrane domain of the CAR, or between the cytoplasmic domain and the transmembrane domain of the CAR. As used herein, the term “linker” generally refers to any oligopeptide or polypeptide that functions to link the transmembrane domain to the extracellular or cytoplasmic domain of a polypeptide chain. The linker may comprise 0-300 amino acids, preferably 2 to 100 amino acids, and most preferably 3 to 50 amino acids.

In a preferred embodiment of the present invention, the extracellular domain of the CAR provided by the present invention includes an antigen binding domain targeting BCMA. The CAR of the present invention, when expressed in T cells, is capable of antigen recognition based on antigen binding specificity. When it binds to its cognate antigen, it affects tumor cells, causing the tumor cells to not grow, to die, or to be otherwise affected, and resulting in a reduction or elimination of the patient's tumor load. The antigen binding domain is preferably fused to one or more intracellular domains of the costimulatory molecule and the ζ chain. Preferably, the antigen binding domain is fused to the 4-1BB signaling domain, and to the intracellular domain of a combination with the CD3ζ signaling domain.

As used herein, “antigen binding domain” and “single chain antibody fragment” both refer to a Fab fragment, Fab′ fragment, F(ab′)₂ fragment, or a single Fv fragment, which has antigen binding activity. The Fv antibody contains an antibody heavy chain variable region, a light chain variable region, but no constant regions, and is the smallest antibody fragment with all antigen-binding sites. Typically, the Fv antibody also contains a polypeptide linker between the VH and VL domains and is capable of forming a structure required for antigen binding. The antigen binding domain is usually a scFv (single-chain variable fragment). The size of scFv is generally ⅙ of that of a complete antibody. The single chain antibody is preferably one amino acid chain sequence encoded by one nucleotide chain. As a preferred mode of the present invention, the scFv comprises an antibody that specifically recognizes BCMA, preferably a single-chain antibody.

For the hinge region and the transmembrane region (transmembrane domain), the CAR can be designed to include a transmembrane domain fused to the extracellular domain of the CAR. In one embodiment, the transmembrane domain naturally associated with one of the domains in the CAR is used. In some examples, the transmembrane domain may be selected, or modified by amino acid substitutions to avoid binding such domains to transmembrane domains of the same or different surface membrane proteins, thereby minimizing the interaction with other members of the receptor complex.

The intracellular domains in the CAR of the present invention include the signaling domain of 4-1BB and the signaling domain of CD3 ζ.

Gene Silencing Methods

At present, commonly used gene silencing methods include CRISPR/Cas9, RNA interference technology, TALENs (transcription activator-like (TAL) effector nucleases) and Zinc finger nucleases (ZFNs), of them, CRISPR/Cas9 has the best prospect and effect of use.

CRISPR (clustered regularly interspersed short palindromic repeats)/Cas (CRISPR-associated) system is a natural immune system unique to prokaryotes and is used to resist the invasion of viruses or exogenous plasmids. Type II CRISPR/Cas systems have been successfully used in many eukaryotes and prokaryotes as tools for direct RNA-mediated genome editing. The development of the CRISPR/Cas9 systems has revolutionized people's ability to edit DNA sequences and regulate the expression levels of target genes, thereby providing a powerful tool for precise genome editing of organisms. The simplified CRISPR/Cas9 system consists of two parts, Cas9 protein and sgRNA. Its principle of action is that the sgRNA forms a Cas9-sgRNA complex with the Cas9 protein through its own Cas9 handle, and the complementary base pairing region sequence of the sgRNA in the Cas9-sgRNA complex and the target sequence of the target gene are paired and bound by the complementary base pairing principle, and Cas9 uses its own endonuclease activity to cleave target DNA sequences. Compared with traditional genome editing techniques, the CRISPR/Cas9 system has several remarked advantages: ease of use, simplicity, low cost, programmability, and allowing editing multiple genes simultaneously.

Vector

A nucleic acid sequence encoding a desired molecule can be obtained using recombinant methods known in the art, for example, by screening libraries from cells expressing the gene, by obtaining the gene from a vector known to include the gene, or by using standard technology to isolate the gene directly from cells and tissues that contain it. Alternatively, the gene of interest can be produced synthetically.

The present invention also provides a vector into which the expression kit of the present invention is inserted. Vectors derived from retroviruses such as lentiviruses are suitable tools for long-term gene transfer because they allow long-term, stable integration of the transgene and the proliferation thereof in its daughter cells. Lentiviral vectors have advantages over vectors derived from oncogenic retroviruses such as murine leukemia viruses because they can transduce non-proliferating cells such as hepatocytes. They also have the advantage of low immunogenicity.

In short, an expression kit or nucleic acid sequence of the present invention is typically operably linked to a promoter and incorporated into an expression vector. The vector is suitable for replicating and integrating eukaryotic cells. Typical cloning vectors include transcriptional and translational terminators, initial sequences and promoters that can be used to regulate the expression of the desired nucleic acid sequence.

The expression constructs of the present invention can also be used in nucleic acid immunization and gene therapy by using standard gene delivery solutions. Methods for gene delivery are known in the art. See, e.g., U.S. Pat. Nos. 5,399,346, 5,580,859, 5,589,466, which are incorporated herein in their entirety by reference. In another embodiment, the present invention provides gene therapy vectors.

The nucleic acid can be cloned into many types of vectors. For example, the nucleic acid can be cloned into vectors including, but not limited to, plasmids, phagemids, phage derivatives, animal viruses, and cosmids. Particular vectors of interest include expression vectors, replication vectors, probe generation vectors, and sequencing vectors.

Further, expression vectors can be provided to cells in the form of viral vectors. Viral vector techniques are well known in the art and are described, for example, by Sambrook et al. (2001, Molecular Cloning: A Laboratory Manual, Cold Spring Harbor Laboratory, New York) and in other manuals of virology and molecular biology. Viruses that can be used as vectors include, but are not limited to, retroviruses, adenoviruses, adeno-associated viruses, herpesviruses, and lentiviruses. Typically, suitable vectors contain an origin of replication functional in at least one organism, a promoter sequence, convenient restriction enzyme sites, and one or more selectable markers (e.g., WO01/96584; WO01/29058; and U.S. Pat. No. 6,326,193).

A number of virus-based systems have been developed for transfer of genes into mammalian cells. For example, retroviruses provide a convenient platform for gene delivery systems. The selected gene can be inserted into a vector and packaged into retroviral particles by using techniques known in the art. The recombinant virus can then be isolated and delivered to subject cells in vivo or ex vivo. Many retroviral systems are known in the art. In some embodiments, adenoviral vectors are used. Many adenoviral vectors are known in the art. In one embodiment, lentiviral vectors are used.

Additional promoter elements, such as enhancers, can regulate the frequency at the start of transcription. Typically, these are located in a region of 30-110 bp upstream of the start site, although it has recently been shown that many promoters also contain functional elements downstream of the start site. The spacing between promoter elements is often flexible so that the promoter function is maintained when elements are inverted or moved relative to one another. In the thymidine kinase (tk) promoter, the spacing between promoter elements can be increased by 50 bp before activity begins to decline. Depending on the promoter, individual elements demonstrate to act cooperatively or independently to initiate transcription.

An example of a suitable promoter is the immediate early cytomegalovirus (CMV) promoter sequence. The promoter sequence is a strong constitutive promoter sequence capable of driving high-level expression of any polynucleotide sequence operably linked to it. Another example of a suitable promoter is elongation growth factor-1α (EF-1α). However, other constitutive promoter sequences can also be used, which include, but are not limited to, the simian virus 40 (SV40) early promoter, mouse mammary tumor virus (MMTV), human immunodeficiency virus (HIV) long terminal repeat (LTR) promoter, MoMuLV promoter, avian leukemia virus promoter, Epstein-Barr virus immediate-early promoter, Rous sarcoma virus promoter, and human gene promoters, for example but not limited to, the actin promoter, myosin promoter, heme promoter and creatine kinase promoter. Further, the present invention should not be limited to the use of constitutive promoters. Inducible promoters are also contemplated as part of the present invention. The use of an inducible promoter provides a molecular that can turn on the expression of a polynucleotide sequence operably linked to the inducible promoter when such expression is desired or turn off the expression when the expression is not desired. Examples of inducible promoters include, but are not limited to, the metallothionein promoter, glucocorticoid promoter, progesterone promoter, and tetracycline promoter.

To evaluate the expression of a CAR polypeptide or portion thereof, the expression vector introduced into cells may also contain either or both of a selectable marker gene and a reporter gene to facilitate the search for identification and selection of expressing cells in the transfected or infected cell population through the viral vector. In other aspects, the selectable marker can be carried on a single section of DNA and used in co-transfection procedures. Both the selectable marker and the reporter gene can be flanked by appropriate regulatory sequences to enable expression in the host cell. Useful selectable markers include, for example, antibiotic resistance genes such as neo and the like.

A reporter gene is used to identify potentially transfected cells and to evaluate the functionality of regulatory sequences. Typically, a reporter gene is a gene that is not present in or expressed by the recipient organism or tissue and that encodes a polypeptide whose expression is clearly indicated by some readily detectable properties such as enzymatic activity. After the DNA has been introduced into the recipient cell, the expression of the reporter gene is measured at an appropriate time. Suitable reporter genes may include genes encoding luciferase, β-galactosidase, chloramphenicol acetyltransferase, secreted alkaline phosphatase, or green fluorescent protein (e.g., Ui-Tei et al., 2000 FEBS Letters 479: 79 -82). Suitable expression systems are well known and can be prepared using known techniques or obtained commercially. Typically, constructs with a minimum of 5 flanking regions showing the highest levels of reporter gene expression are identified as promoters. Such promoter regions can be linked to a reporter gene and used to evaluate the ability of an agent to regulate promoter-driven transcription.

Methods for introducing and expressing genes into cells are known in the art. In terms of the content of an expression vector, the vector can be easily introduced by any method known in the art into a host cell, e.g., mammalian, bacterial, yeast or insect cells. For example, an expression vector can be transferred into a host cell by physical, chemical or biological means.

Physical methods for introducing polynucleotides into host cells include calcium phosphate precipitation, lipofection, particle bombardment, microinjection, electroporation, and the like. Methods for producing cells comprising a vector and/or exogenous nucleic acids are well known in the art. See, e.g., Sambrook et al. (2001, Molecular Cloning: A Laboratory Manual, Cold Spring Harbor Laboratory, New York). The preferred method for introducing polynucleotides into host cells is calcium phosphate transfection.

Biological methods for introducing polynucleotides of interest into host cells include the use of DNA and RNA vectors. Viral vectors, especially retroviral vectors, have become the most widely used method of inserting genes into mammalian, e.g., human cells. Other viral vectors can be derived from lentiviruses, poxviruses, herpes simplex virus I, adenoviruses and adeno-associated viruses, etc. See, e.g., U.S. Pat. Nos. 5,350,674 and 5,585,362.

Chemical means of introducing polynucleotides into host cells include colloidal dispersion systems, such as macromolecular complexes, nanocapsules, microspheres, beads; and lipid-based systems, including oil-in-water emulsions, micelles, mixed micelles, and lipids plastid. Exemplary colloidal systems for use as in vitro and in vivo delivery vehicles are liposomes (e.g., artificial membrane vesicles).

Where non-viral delivery systems are used, exemplary delivery vehicles are liposomes. The use of lipid formulations is contemplated for introducing a nucleic acid into the host cell (in vitro, ex vivo, or in vivo). In another aspect, the nucleic acid can be associated with lipids. The nucleic acid associated with lipids can be encapsulated into the aqueous interior of liposomes, interspersed in the lipid bilayer of liposomes, attached to liposomes via a linker molecule associated with both the liposomes and oligonucleotides, entrapped in liposomes, complexed with liposomes, dispersed in lipid-containing solutions, mixed with lipids, associated with lipids, contained in lipids as a suspension, contained in micelles or complex with micelles, or otherwise associated with lipids. The lipids, lipids/DNA or lipids/expression vector associated with the composition are not limited to any particular structure in a solution. For example, they may exist in bilayer structures, as micelles or have a “collapsed” structure. They can also simply be dispersed in a solution, possibly forming aggregates that are not uniform in size or shape. Lipids are fatty substances, which can be naturally occurring or synthetic lipids. For example, lipids include lipid droplets, which occur naturally in the cytoplasm as well as in such compounds comprising long chain aliphatic hydrocarbons and their derivatives such as fatty acids, alcohols, amines, amino alcohols and aldehydes.

In a preferred embodiment of the present invention, the vector is a lentiviral vector.

Formulations

The present invention provides a CAR-T cell of the first aspect of the present invention, and a pharmaceutically acceptable carrier, diluent or excipient. In one embodiment, the formulation is a liquid formulation. Preferably, the formulation is an injection. Preferably, the concentration of the CAR-T cells in the preparation is 1×10³-1×10⁸ cells/ml, more preferably 1×10⁴-1×10⁷ cells/ml.

In one embodiment, the formulation may include a buffer such as neutral buffered saline, sulfate buffered saline, etc.; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives. The formulations of the present invention are preferably formulated for intravenous administration.

Therapeutic Use

The present invention includes therapeutic use of the CAR-T cells described in the first aspect of the present invention. The transduced T cells can target the tumor cell marker BCMA, synergistically activate T cells, and cause T cell immune responses, thereby significantly improving their killing efficiency against tumor cells.

Accordingly, the present invention also provides a method of stimulating a T cell-mediated immune response to a target cell population or tissue in a mammal, comprising a step of administering the CAR-T cells of the present invention to the mammal.

In one embodiment, the present invention includes a cell therapy, wherein a patient's autologous T cells (or allogeneic donor) are isolated, activated and genetically engineered to produce CAR-T cells, which are then infused into the patient. In this way, the probability of graft-versus-host disease is extremely low, and the antigen is recognized by T cells in an MHC-unrestricted way. In addition, one CAR-T can treat all cancers that express this antigen. Unlike antibody therapies, CAR-T cells can replicate in vivo, resulting in long-lasting sustained tumor control.

In one embodiment, the CAR-T cells of the present invention can have robust in vivo proliferation of T cells for an extended length of time. Additionally, a CAR-mediated immune response can be part of an adoptive immunotherapy step in which CAR-modified T cells induce an immune response specific to the antigen binding domain in the CAR. For example, anti-BCMA CAR-T cells induce specific immune responses against BCMA-expressing cells.

Although the data of this disclosure specifically discloses lentiviral vectors comprising anti-BCMA scFv, hinge and transmembrane regions, and 4-1BB and CD3ζ signaling domains, the present invention should be construed as including any variation in the number of each of the components of the construct.

Cancers that can be treated include tumors that are not vascularized or not substantially vascularized, as well as tumors that are vascularized. Cancers may include non-solid tumors (such as hematological tumors, e.g., leukemias and lymphomas) or may include solid tumors. Cancer types that are treated with the CARs of the present invention include, but are not limited to, carcinomas, blastomas, and sarcomas, and certain leukemic or lymphoid malignancies, benign and malignant tumors, and malignant tumors, such as sarcomas, carcinomas, and melanomas. Adult tumors/cancers and childhood tumors/cancers are also included.

Hematological cancers are cancers of the blood or bone marrow. Examples of hematological (or hematogenous) cancers include leukemias, including acute leukemias (such as acute lymphoblastic leukemia, acute myeloid leukemia, acute myeloid leukemia, and myeloblastoid, promyelocytic, granulocytic, monocytic and erythroleukemia), chronic leukemia (such as chronic myeloid (myeloid) leukemia, chronic myelogenous leukemia, and chronic lymphocytic leukemia), polycythemia vera, lymphoma, Hodgkin's disease, non-Hodgkin's lymphoma (painless and high-grade forms), multiple myeloma, Waldenstrom's macroglobulinemia, heavy chain disease, myelodysplastic syndrome, hairy cell leukemia, and myelodysplasia.

Solid tumors are abnormal masses of tissue that typically do not contain cysts or fluid regions. Solid tumors can be benign or malignant. Different types of solid tumors are named after the cell type that forms them (such as sarcomas, carcinomas, and lymphomas). Examples of solid tumors such as sarcomas and carcinomas include fibrosarcoma, myxosarcoma, liposarcoma, mesothelioma, lymphoid malignancies, pancreatic cancer, and ovarian cancer.

The CAR-modified T cells of the present invention can also be used as a type of vaccine for ex vivo immunization and/or in vivo therapy of mammals. Preferably, the mammal is a human.

For ex vivo immunization, at least one of the following occurs in vitro prior to administering the cells into a mammal: i) proliferating cells, ii) introducing the nucleic acid encoding the CAR into the cells, and/or iii) cryopreserving the cells.

Ex vivo procedures are well known in the art and are discussed more fully below. Briefly, cells are isolated from mammals (preferably humans) and genetically modified (i.e., transduced or transfected in vitro) with a vector expressing the CAR disclosed herein. The CAR-modified cells can be administered to mammalian recipients to provide therapeutic benefits. The mammalian recipient can be human, and the CAR-modified cells can be autologous to the recipient. Alternatively, the cells may be allogeneic, syngeneic or xenogeneic to the recipient.

In addition to using cell-based vaccines for ex vivo immunization, the present invention also provides compositions and methods for in vivo immunization to elicit an immune response against antigens in patients.

The present invention provides methods of treating tumors comprising administering to a subject in need thereof a therapeutically effective amount of the CAR-modified T cells of the present invention.

The CAR-modified T cells of the invention can be administered alone or as a pharmaceutical composition in combination with diluents and/or with other components such as IL-2, IL-17 or other cytokines or cell populations. Briefly, the pharmaceutical compositions of the present invention may include the target cell populations as described herein, in combination with one or more pharmaceutically or physiologically acceptable carriers, diluents or excipients. Such compositions may include buffers such as neutral buffered saline, sulfate buffered saline, and the like; carbohydrates such as glucose, mannose, sucrose or dextran, mannitol; proteins; polypeptides or amino acids such as glycine; antioxidants; chelates such as EDTA or glutathione; adjuvants (e.g., aluminum hydroxide); and preservatives. The compositions of the present invention are preferably formulated for intravenous administration.

The pharmaceutical compositions of the present invention can be administered in a way appropriate to the disease to be treated (or prevented). The amount and frequency of administration will be determined by factors such as the patient's condition, and the type and severity of the patient's disease, although appropriate dosages can be determined by clinical trials.

When referring to an “immunologically effective amount”, “effective amount against tumors”, “effective tumor-suppressing amount” or “therapeutic amount”, the precise amounts of the compositions of the invention to be administered can be determined by physicians, taking into account the individual differences of patients (subjects) in terms of age, weight, tumor size, degree of infection or metastasis, and medical condition. It may generally be noted that the pharmaceutical compositions comprising the T cells described herein may be administered at doses ranging from 10⁴ to 10⁹ cells/kg body weight, preferably 10⁵ to 10⁶ cells/kg body weight (including all integer values within those ranges). The T cell compositions can also be administered multiple times at these doses. The cells can be administered using infusion techniques well known in immunotherapy (see, e.g., Rosenberg et al., NewEng. J. of Med. 319: 1676, 1988). The optimal dosage and treatment regimen for a particular patient can be easily determined by those skilled in the medical arts by monitoring the patient for signs of disease and adjusting treatment accordingly.

Administration of the compositions to subjects can be carried out in any convenient way, including by nebulization, injection, swallowing, infusion, implantation, or transplantation. The compositions described herein can be administered to patients subcutaneously, intradermally, intratumorally, intranodal, intraspinal, intramuscularly, by intravenous (i.v.) injection, or intraperitoneally. In one embodiment, the T cell compositions of the present invention are administered to patients by intradermal or subcutaneous injection. In another embodiment, the T cell compositions of the present invention are preferably administered by i.v. injection. The T cell compositions can be injected directly into tumors, lymph nodes or sites of infection.

In some embodiments of the present invention, the T cells activated and amplified to therapeutic levels by using the methods described herein, or other methods known in the art, are combined with any number of relevant therapeutic modalities (e.g., before, concurrently or after) to patients, the therapeutic modalities including but not limited to, treatment with agents such as antiviral therapy, cidofovir and interleukin-2, cytarabine (also known as ARA-C) or natalizumab therapy for MS patients or elfazizumab therapy for psoriasis patients or other treatments for PML patients. In a further embodiment, the T cells of the present invention may be used in combination with chemotherapy, radiation, immunosuppressive agents such as cyclosporine, azathioprine, methotrexate, mycophenolate mofetil and FK506, antibodies or other immunotherapeutic agents. In a further embodiment, the cell compositions of the present invention are administered (e.g., before, concurrently or after) to patients in combination with bone marrow transplantation, use of chemotherapeutic agents such as fludarabine, external beam radiation therapy (XRT), and cyclophosphamide. For example, in one embodiment, the subject may undergo standard treatment with high dose chemotherapy followed by peripheral blood stem cell transplantation. In some embodiments, after transplantation, the subject receives infusions of the amplified immune cells of the present invention. In an additional embodiment, the amplified cells are administered before or after surgery.

The dosages of the above treatments administered to patients will vary depending on the precise nature of the condition being treated and the recipients of the treatments. Dosage ratios for human administration can be carried out in accordance with the accepted practice in the art.

Typically, for each treatment or each course of treatment, 1×10⁶ to 1×10¹⁰ modified T cells of the present invention (e.g., CAR-T20 cells) can be administered to a patient, e.g., by intravenous infusion.

Main Advantages of the Present Invention Include:

(a) The present invention provides Tscm-enriched second-generation universal CAR-T cells, which show better amplification rate, phenotype, IFN-γ releasing capacity and target cell killing capacity than the first generation. Based on the Tscm-enriched T cells of the present invention (especially the second-generation universal CAR-T cells), highly active universal CAR-T cells against different targets can be prepared.

(b) The present invention also prepares universal T cells targeting tumors (for example, universal T cells of C-CAR088 targeting BCMA), which can specifically recognize and kill target cells that express respective tumor targets (such as BCMA tumor antigens).

(c) The present invention provides a method for preparing Tscm-enriched T cells, which can efficiently prepare Tscm-enriched T cell populations, especially universal CAR-T cell populations.

The present invention will be further described below in conjunction with specific embodiments. It should be understood that these embodiments are used only to illustrate the present invention and not to limit the scope of the present invention. The experiment method with unindicated specific conditions in the following embodiments are usually performed according to conventional conditions, e.g., Sambrook et al., Molecular Cloning: Laboratory Manual (New York: Cold Spring Harbor Laboratory Press, 1989), or as suggested by the manufacturer. Percentages and parts are by weight unless otherwise indicated.

General Experiment Methods

1. Lentiviral Preparation of BCMA-CAR

In the present invention, BCMA is used as a representative tumor-related specific target to prepare CAR-T cells targeting the tumor cells.

On day 0, put 5×10⁶ 293T cells in 10 ml of polylysine-pretreated DMEM medium in a 10-cm dish. Incubated the cells overnight at 37° C. On day 1, prepared the transfection reagent.

Added 45.5 ul of lipofectamine 3000 reagent to OMEM until the total volume of the mixture reached 500 ul and incubated for 5 minutes at room temperature. Prepared another OMEM sample with a total volume of 500 ul and added 10.7 ug BCMA-CAR lentiviral vector, 34 ug PRP2 plasmid, 1.8 ug PRP3 plasmid, 2.3 ug PRM2 plasmid and 36.4 ul P3000 reagent. Mixed the two reagents in a 1.5 ml tube and incubated at room temperature for 15 minutes. Added all the transfection reagents to the 293T cell culture, and then incubated the cells at 37° C. for 6 hours. Discarded the complete medium and added 10 ml of pre-warmed DMEM medium. Incubated the cells at 37° C. for 48 hours. On day 3, transferred the culture supernatant to a 15 ml tube and centrifuged with 2000 g for 10 minutes at room temperature. Carefully aliquoted 1 ml of the supernatant into 1.5 ml tubes and stored them at −80° C. for use.

2. Stimulation of T Cells with Anti-CD3 (Clone OKT3) Antibody (day 3)

Added 500 ul PBS containing 5 ug/ml fibronectin and 5 ug/ml anti-CD3 antibody to the wells of a 24-well plate and incubated at 37° C. for 3 hours. Took out the PBS, and adequately washed the plate twice with 1 ml PBS. The plate was ready for T cell stimulation. Counted the

T cells and added 5 x 10⁶ of the T cells each well and centrifuged with 100 g for 5 minutes at room temperature. Discarded the supernatant, resuspended the cells in 2.5 ml AIM-V medium, and then transferred to plates coated with anti-CD3 antibody, and incubated the plates at 37° C. for 72 hours.

3. Activation of T Cells with CD3/CD28 Magnetic Beads

Mixed Dynabeads Human T-Activator CD3/CD28 magnetic beads and the cells in AIM-V medium at 1:1 to activate the T cells. After 72 hours, removed the magnetic beads with magnetic force.

4. Cell Culture

Tscm cell culture: cultured in AIM-V medium containing 10% FBS, Glutamax, Pen/Strip, 10 ng/ml IL15, 5 ng/ml IL7 and 10 ng/ml IL21, at a density of approximately 1×10⁶ cells/ml.

Traditional T cell culture: cultured in AIM-V medium supplemented with 10% FBS, Glutamax, Pen/Strip, 200 IU/ml IL2, at a density of 10⁶ cells/ml.

5. T Cell Nucleofection (Day 0)

In a PCR tube, 90 pmol Alt-R CRISPR_Cas9 crRNA antiTRAC (AGA GUC UCU CAG CUG GUA CAG UUU UAG AGC UAU GCU, SEQ ID NO.: 1) and 90 pmol Alt-R CRISPR-Cas9 tracRNA (AGC AUA GCA AGU UAA AAU AAG GCU AGU CCG UUA UCA ACU UGA AAA GUG GCA CCG AGU CGG UGC UUU U, SEQ ID NO.: 2) were mixed.

In another PCR tube, 90 pmol Alt-R CRISPR-Cas9 crRNA antiB2M (GGC CAC GGA GCG AGA CAU CUG UUU UAG AGC UAU GCU, SEQ ID NO.: 3) and 90 pmol Alt-R CRISPR-Cas9 tracRNA were mixed.

Incubated at 95° C. for 5 minutes and then at 37° C. for 25 minutes. After the reaction, thoroughly mixed the two reagents in a test tube. Added 9 ug Cas9 protein and incubated for 10 minutes at room temperature. Centrifuged 1×10⁶ activated T cells with 100 g for 10 minutes at room temperature. Removed the supernatant, and resuspended the cells in 20 ul nucleofection buffer containing 16.4 ul P3 buffer and 3.6 ul supplemented buffer. Added the cas9 RNP complex prepared in step 12 and mixed well. Transferred the samples to strips for nucleofection and transfected the T cells with the nucleofection program CA137. After the transfection process was completed, added 80 ul AIM-V medium to the strips, then transferred the sample to wells of a 24-well plate, and added AIM-V medium to a final total volume of 1 ml. Incubated the plate at 37° C. for 2 hours and then prepared for viral transduction.

6. BCMA-CAR Lentiviral Transduction of T Cells.

Thawed the previously prepared frozen BCMA-CAR lentiviral supernatant in a 37° C. water bath until fully thawed. Transferred 1 ml BCMA-CAR lentiviral supernatant to the T cell culture, the final total volume of the medium being 2 ml and added protamine sulfate to a final concentration of 1 ug/ml. Mixed them well by pipetting up and down 5 times. Centrifuged the cells at 32° C. with 2000 g for 2 hours, and then transferred the plates to a 37° C. incubator for overnight culture. On day 1, carefully took 1.5 ml of the supernatant, and added 1.5 ml of fresh AIM-V medium. Depending on the cell growth conditions, changed the medium to maintain a cell density of at least 1×10⁶ cells/ml.

7. T Cell Purification (Usually on Days 6-8)

Transferred 4×10⁶ T cells to a 1.5 ml tube and centrifuged with 100 g for 10 minutes at room temperature. Discarded the supernatant, and then resuspended the cells in 100 ul PBS containing 2% FBS. Added 2 ul biotin-conjugated anti-HLA A/B/C and 2 ul biotin-conjugated anti-CD3 antibody to the sample and incubated at 4° C. for 30 minutes. Added 4 ul biotin-separated mixture to the sample and incubated for 15 minutes at room temperature. Added 5 ul rapidspheres and incubated the sample for 10 minutes at room temperature. Transferred the sample to a 5 ml tube, and then added 1.9 ml AIM-V medium. Placed the tube on a magnet and waited for 5 minutes. Transferred the supernatant containing T cells to wells of a 24-well plate.

8. FACS Analysis

(a). Transferred 1×10⁵ T cells to a 1.5 ml tube and centrifuged at 1500 rpm for 10 minutes at room temperature. Discarded the supernatant, then added 900 ul PBS, and centrifuged at 1500 rpm for 10 minutes at room temperature. Discarded the supernatant, resuspended the cells in 100 ul PBS containing 1 ug/ml recombinant human BCMA Fc chimeric protein and incubated on ice for 1 hour. Added 900 ul PBS, centrifuged at 1500 rpm for 10 minutes at room temperature, and then discarded the supernatant. Resuspended the cells in PBS (containing 1:1000 Live/Dead aqua orange staining dye). Incubated on ice for 15 minutes. Added 900 ul PBS, centrifuged at 1500 rpm for 10 minutes at room temperature, and then discarded the supernatant. Resuspended the cells in 100 ul PBS, 100 ul PBS containing 2.5 ul APC-Cy7-conjugated anti-CD3 antibody and 2.5 ul V605-conjugated anti-HLA A/B/C antibody and 1; 100 APC-conjugated anti-BCMA 1 Fc chimeric protein antibody, and incubated on ice for 30 minutes. Added 900 ul PBS, centrifuged at 1500 rpm for 10 minutes at room temperature, and then discarded the supernatant. Resuspended the cells in 300 ul PBS and transferred the sample to a 5 ml tube for FACS analysis.

The T-cell population was firstly grouped by FSC and SSC. An Aqua orange channel was used to distinguish live and dead cells, and an APC channel was used to distinguish BCMA-CAR positive cells. CD3 and HLA A/B/C were used to distinguish CD3 and HLA A/B/C double knockout T cells.

(b). Transferred 1×10⁵ T cells to a 1.5 ml tube and centrifuged at 1500 rpm for 10 minutes at room temperature. Discarded the supernatant, then added 900 ul PBS, and centrifuged at 1500 rpm for 10 minutes at room temperature. Discarded the supernatant and resuspended the cells in PBS (containing 1:1000 Live/Dead aqua orange staining dye). Incubated on ice for 15 minutes. Added 900 ul PBS, centrifuged at 1500 rpm for 10 minutes at room temperature, and then discarded the supernatant. Resuspended the cells in 100 ul PBS, containing 2.5 ul APC-conjugated anti-CCR7 antibody, incubated at 37 degrees for 30 minutes, and then added 2.5 ul PE-Cy7-conjugated anti-CD45RA antibody, 2.5 ul FITC-conjugated anti-CD8 antibody and 2.5 ul APC-Cy7-conjugated anti-CD3 antibody, and incubated on ice for 30 minutes. Added 900 ul PBS, centrifuged at 1500 rpm for 10 minutes at room temperature, and the discarded the supernatant. Resuspend the cells in 300 ul PBS and transferred the sample to a 5 ml tube for FACS analysis.

9. ELISA

On the day before, used Human IFN-γ Mab to coat ELISA plates. Diluted Human IFN-γ Mab with 1×PBS (1:1000) to 1 ug/mL, added 100 ul of the antibody to each well, and sealed the plates with a sealing film, and kept them at 4° C. overnight. Quickly poured out the liquid in the plates, added 100 ul Assay Buffer to each well, covered with a sealing film, and sealed at room temperature for 30 minutes. Washing the plates: quickly poured out the liquid in the plates, added washing buffer, 200 ul per well, with a discharge gun, and repeated the washing of the plates one more time. Prepared Human IFN-γ Biotin-labeled Mab, diluted it with Assay Buffer dilution (1:500), and added it to the plates 50 ul per well. Prepared Human IFN-γ ELISA Standard, set 8 gradients (in pg/ml): 2000, 1000, 500, 250, 125, 62.5, 31.25, 0.

Added the samples and standards to the ELISA plate, 100 ul/well, used Assay Buffer to dilute both the samples and the standards to the desired concentration, covered with a sealing film, and incubated at room temperature for 1.5 hours. Wash the plates: quickly poured out the liquid in the plates, added washing buffer with a discharge gun, 200 ul per well, and repeated the washing of the plates four times. Prepared HRP-conjugated streptavidin and used Assay Buffer to dilute it (1:5000), and added it, 100 ul per well, to the plate. Covered with a sealing film and incubated at room temperature for 30 minutes. Wash the plates: quickly poured out the liquid in the plates, added washing buffer with a discharge gun, 200 ul per well, and repeated the washing two times. Returned the TMB substrate to room temperature 30 minutes beforehand, and added it, 100 ul each well, to the plates. After 5-10 minutes of reaction at room temperature, added the terminating solution, 50 ul/well. Detected the absorbance at the 450 nm wavelength with an ELISA reader, generated a standard curve according to the concentration of the standard and the OD values, and calculated the IFN-γ concentration of the test sample based on the OD values of the test sample.

10. RTCA Cell Killing Real-Time Analysis

Day 1: Opened THE RTCA MP operating software and entered experiment information. Took a 96-well E-plate culture plate that worked with RTCA and added 50 ul F12 complete medium to each well, avoiding air bubbles. Placed the plate into the RTCA instrument, and the operating software read the background before the cells were added. Confirmed that the reading was normal. The A549 and A549-BCMA cells cultured by trypsinization were counted for live cells by using the AO/PI Cell Viability kit, and the live cells were centrifuged, and the two tumor cells were resuspended to 4×10⁵/ml in F12 complete medium. According to the experimental arrangement, added 50 ul of the cell suspension (i.e., 2×10⁴ A549 and A549-BCMA cells) to each well of the E-plate whose background had been read. Set 3 replicate wells for each group of experiments to ensure that they included 3 wells of the control group, that is, containing only the tumor cells without adding T cells subsequently. The plates were placed in an ultraclean station for 30 minutes to allow the cells to adhere to the wall evenly. Put the plates into the RTCA instrument, and the operating software started to record the cell index (Cell Index) changes, being set to take reading every 15 minutes, and ensured that the total recording length was set to more than 4 days.

Day 2: The T cells were counted and centrifuged, and then the T cells were resuspended in T cell medium and diluted to different concentrations of 1×10⁵/ml, 5×10⁴/ml, 2.5×10⁴/ml, 1.25×10⁴/ml. Paused the software recording of RTCA, took the E-plate from the instrument to an ultraclean station, and added 100 ul 1×10 ⁵ ml of the T cell suspension to the A549 culture well, that is, the ratio of the final T cells to the target cells was 0.5:1; added 100 ul 1×10⁵/ml, 5×10⁴/ml, 2.5×10⁴/ml, 1.25×10⁴/ml of the T cell suspension to the A549-BCMA culture well, that is, the final ratio of the T cells to the target cells was 0.5:1, 0.25:1, 0.125:1, 0.0625:1. Returned the E-plate to the RTCA instrument and read the cell index every 15 min until the set end time. After the experiment, data analysis was performed. Generally, the last time point before the T cells were added was used as a reference, and the reading of the tumor cells only without adding T cells was set as 100%, and the changes in the tumor cell index after adding the T cells were converted to a killing curve.

### EXAMPLE 1

### Analysis of TRAC and B2M Gene Knockout and Purification Rates in Universal T Cells

Knockout of TRAC resulted in the disappearance of CD3 expression on the cell surface, and knockout of B2M resulted in the disappearance of HLAA/B/C expression on the cell surface. Therefore, anti-HLA A/B/C and anti-CD3 antibodies were used to stain the genetically edited T cells and a flow cytometric analysis was performed to detect the TRAC and B2M knockout rates.

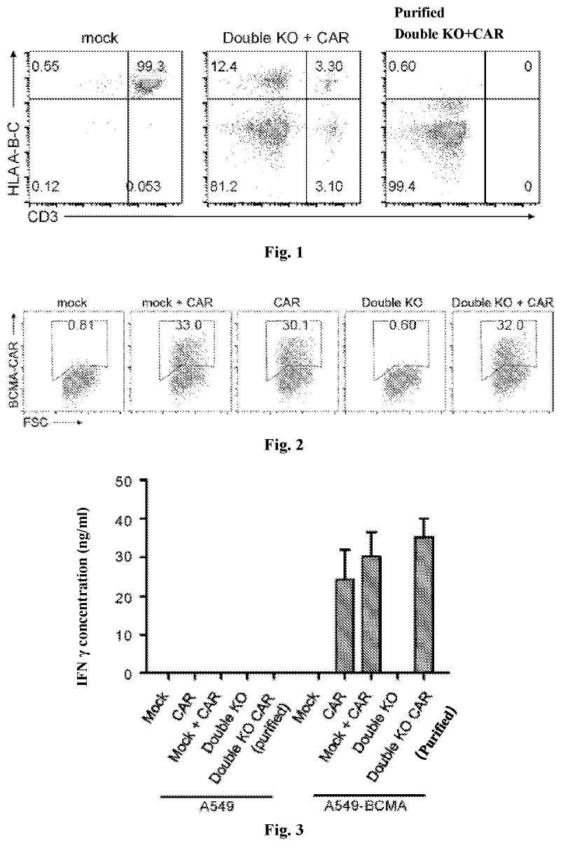

The results are shown in FIG. 1, the double knockout rate of the TRAC and B2M genes in the T cells on day 7 after the Cas9 RNP gene editing and BCMA CAR transduction was 81.2%. After purification, the double knockout cells were enriched from 81.4% to 99.4%, while a small amount of T cells with low HLA A/B/C expression were present under the HLA A/B/C gate line.

### EXAMPLE 2

### Efficiency of BCMA CAR Transduction of Universal T Cells

A flow cytometric analysis of BCMACAR-transduced T cells was performed on day 7.

The results are shown in FIG. 2, and the BCMA CAR transduction efficiency was similar between the groups (mock: T cells without Cas9 RNP nucleofection; mock+CAR: T cells without Cas9 RNP nucleofection but transduced with BCMA CAR; CAR: T cells transduced with BCMACAR; Double KO: T cells with TRAC and B2M double knockout after Cas9 RNP nucleofection; Double KO+CAR: T cells of the above-mentioned Double KO cells after transduction with BCMA CAR).

The above results show that the gene editing process of nucleofection did not affect the CAR transduction efficiency.

### EXAMPLE 3

### Responsiveness of Universal BCMA CAR-T Cells to Target Cells

Used ELISA to detect the IFN-γ levels in the supernatants after co-culture of the above-mentioned different BCMA CAR-T cells and the target cells or BCMA antigen-expressing A549-BCMA cells.

The results are shown in FIG. 3, and Double KO+CAR, i.e., genetically edited BCMA CAR-T cells showed responsiveness to target cells, i.e., IFN-γ releasing capacity, similar to other unedited conventional BCMA CAR-T cells.

The above results show that: the universal CAR-T cells and autologous CAR-T cells have similar functions.

### EXAMPLE 4

### Preparation of the Second-Generation Universal CAR-T Cells

Naive T cells (Tn) were enriched from peripheral blood, and a flow cytometric analysis was performed to examine the efficiency of CCR7⁺CD45RA⁺-enriched naive T cells (Tn).

4.1 Enrichment of Tn Cells from Peripheral Blood The method is as follows: Used cytokine-free AIM-V medium to thaw human PBMCs and counted the cells. Centrifuged the cells (300 g, 5 min), removed the supernatant, and resuspended the cells in PBS in a 5 ml tube. Added biotin isolation cocktail and anti-TCRγ/δ antibody and incubated for 5 minutes at room temperature. Added rapidSpheres and incubated for 3 minutes at room temperature. Added cytokine-free AIM-V medium to 2.5 ml, placed it on a magnetic stand and waited for 3 minutes. Transferred the supernatant to a new 5 ml tube, placed it on the magnetic stand and waited for 3 minutes. The Tn cell-containing supernatant was transferred to a new 15 ml tube, and then the cells were counted.

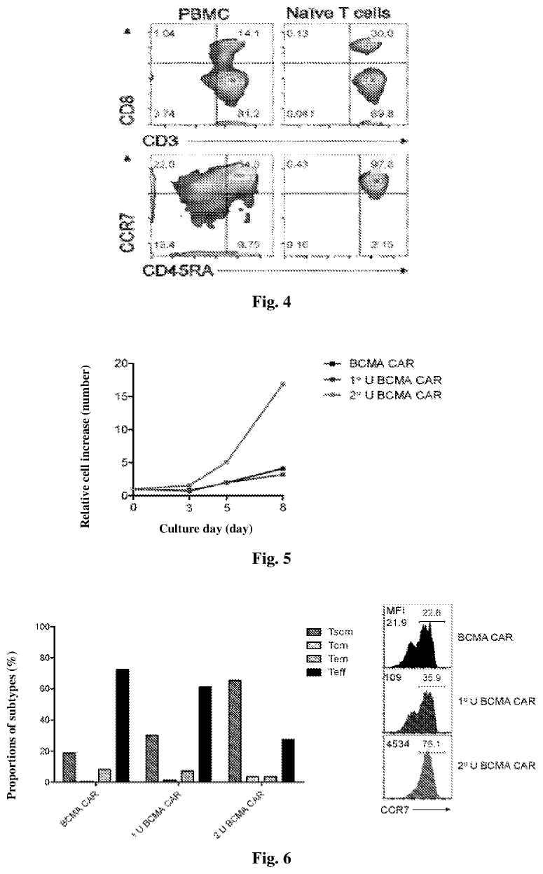

The results are shown in FIG. 4. After enrichment and purification, the proportion of Tn increased from 54.8% in PBMC to 97.3%.

If necessary, the enriched and purified Tn cells can be amplified and cultured, for example, in AIM-V medium containing 10% FBS, Glutamax, Pen/Strip, 10 ng/ ml IL15, 5 ng/ml IL7 and 10 ng/ml IL21 at about 10⁶ cells/ml.

4.2 Preparation of the Second-Generation Universal CAR-T Cells

Repeated those in Examples 1-3 except that: the conventional T cells were replaced by highly purified Tn cells prepared from 4.1.

The TRAC and B2M genes were knocked out from the highly purified Tn cells prepared in 4.1, and then lentivirus transduction was performed to introduce BCMA CAR to prepare the second-generation universal CAR-T cells.

4.3 Preparation of the Second-Generation Universal CAR-T Cells by Direct Amplification Method (the Operation of Enriching Tn is Omitted)

Another method for preparing the second-generation universal CAR-T cells starts from the total T cells (Total T cells) enriched from peripheral blood, but in the process of their amplification and culture, they are cultured under a suitable condition (e.g., in AIM-V medium containing 10% FBS, Glutamax, Pen/Strip, 10 ng/ml IL15, 5 ng/ml IL7 and 10 ng/ml IL21) at a density of about 10⁶ cells/ml, thereby obtaining a Tn-enriched T cell population (under this condition, Tn cells will amplify more preferentially and efficiently).

Next, gene knockout (such as knockout of TRAC and/or B2M), lentiviral transduction and other steps are performed to obtain Tscm-enriched universal CAR-T cells, that is, the second-generation universal CAR-T cells.

### EXAMPLE 5

### Amplification Efficiency of the Second-Generation Universal T Cells

The second-generation universal T cells prepared in Example 4.2 were counted. The results are shown in FIG. 5, and the second-generation universal T cells showed higher amplification efficiency than the other two CAR-T cells, having a relative increase rate up to 16 times on day 7, about 3-4 times greater than the other two CARs, which indicates that the culture condition for preparing the second-generation universal T cells is more favorable for the proliferation of CAR-T cells than the traditional condition for preparing CARs.

### EXAMPLE 6

### The Proportion of Each T Cell Subtype in the Second-Generation Universal CAR-T Cell Product

A flow cytometric phenotype analysis was performed for the second-generation universal CAR-T cell product prepared in Example 4.2.

The results are shown in FIG. 6, and the proportion of Tscm in the second-generation universal CAR-T cell product is close to 70%, which is much higher than those in the first-generation universal CAR-T cell product (about 30%) and autologous CAR-T cells (about 20%).

Unexpectedly, in the second-generation universal CAR-T cells, the percentage of the biomarker CCR7 for de/weakly-differentiated cells was as high as 76%. In addition, the MFI of the second-generation universal CAR-T cells reached 4534, which was much higher than the corresponding MFI values of the first-generation universal CAR-T cell products and autologous CAR-T cells.

The above results show that the conditions for preparing the second-generation universal CAR-T cells of the present invention are conducive to the production of a high proportion of de/weakly-differentiated CAR-T cells, that is, Tscm-enriched CAR-T cells, and the generated CAR-T cells have higher viability and killing activity.

### EXAMPLE 7

### Efficiency of BCMACAR Transduction of the Second-Generation Universal T Cells

A flow cytometric analysis of the CAR expression rate was performed for the second-generation universal CAR-T cell product prepared in Example 4.2.

The results are shown in FIG. 7, and the proportion of BCMA CAR in the second-generation universal CAR-T cell product reached 83.4%, which was higher than those in the first-generation universal CAR-T cell product (60.8%) and in autologous CAR-T cells (60.2%).

The above results show that the second-generation universal CAR-T cells prepared by the present invention did not reduce or even increase the transduction rate of CAR.

### EXAMPLE 8

### Responsiveness of the Second-Generation Universal BCMA CAR-T Cells to Target Cells

Used ELISA to detect by of IFN-γ level in the supernatant after co-culture of the above-mentioned different BCMA CAR-T cells and BCMA antigen-expressing target cells or A549-BCMA cells.

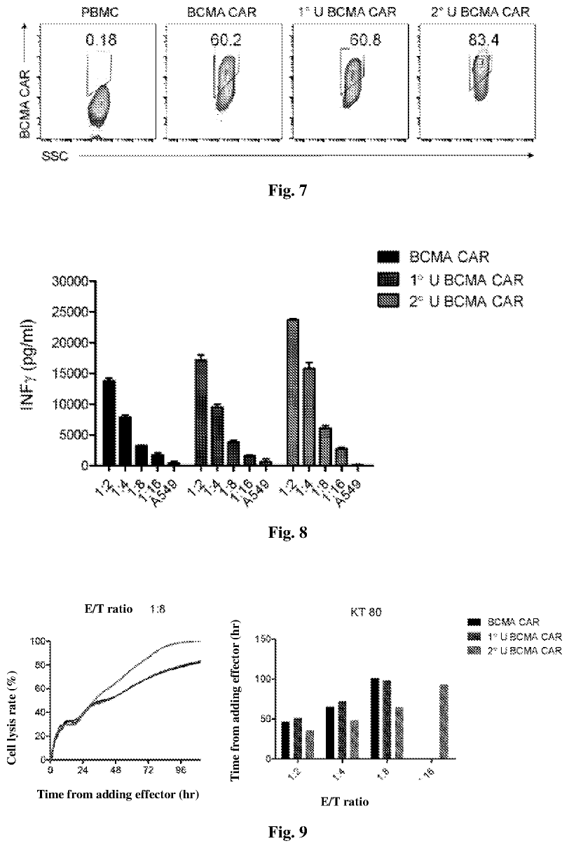

The results are shown in FIG. 8, and the second generation BCMA CAR-T cells have significantly higher responsiveness to the target cells, i.e., IFN-γ releasing capacity, than the first-generation universal BCMA CAR-T cells and autologous BCMA CAR-T cells.

In addition, at different effector cell: target cell ratios (from 1:2 to 1:16), the second-generation universal CAR-T cells responded significantly better to the target cells than the other two CAR-T cells. This suggests that the second-generation universal CAR-T cells of the present invention more remarkedly respond to target cells with corresponding targets (e.g., tumor antigens, such as BCMA or other targets).

### EXAMPLE 9

### The Killing Power of the Second-Generation Universal BCMA CAR-T Cells Against the Target Cells

When a RTCA platform was used to continuously monitor the above 3 kinds of BCAM CAR-T cells for the killing of the target cells for 4 days.

The results are shown in FIG. 9, and echoing the results of the above IFN-γ release experiments, the second generation of universal BCMA CAR-T cells have the best killing power against the target cells. The cell lysis curve on the left side of FIG. 9 shows that the second generation of universal BCMA CAR-T cells have higher killing power against the target cells than the other two CAR-T cells. Shown here is an example of the 1:8 ratio of CAR-T cells to target cells, but the same conclusions apply to other 1:2, 1:4, and 1:16 cases (data not shown).

From the KT80 diagram on the right side of FIG. 9, that is, the diagram of time required to kill 80% of the target cells, it can be seen that, at different ratios of CAR-T cells and target cells, the second-generation universal BCMA CAR-T cells are significantly lower in the time required to kill 80% of the target cells than the other two CAR-T cells. For example, when the number of CAR-T cells to the number of target cells is 1:8, it takes about 70 hours for the second-generation universal cells to kill 80% of the target cells, but it takes about 100 hours for the other two types of CAR-T cells, about 1.5 times the time required when using second-generation CAR-T cells.

In the case where the number of CAR-T cells is much smaller than the target cells (1:16), the other two CAR-T cells cannot even achieve the killing of 80% of the target cells, only the second-generation universal BCMA CAR-T cells can achieve this killing power. The above results show that the second-generation universal CAR-T cells are more effective in killing the target cells than the other two types of CAR-T cells, especially when targeting a large number of the target cells.

### EXAMPLE 10

In this example, the amplification and culture of T cells under different conditions were tested, and except that the added cytokines were changed compared to Example 4, other culture conditions were the same.

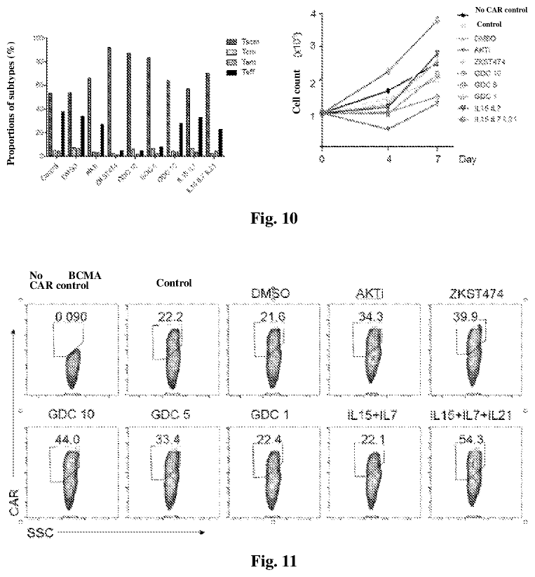

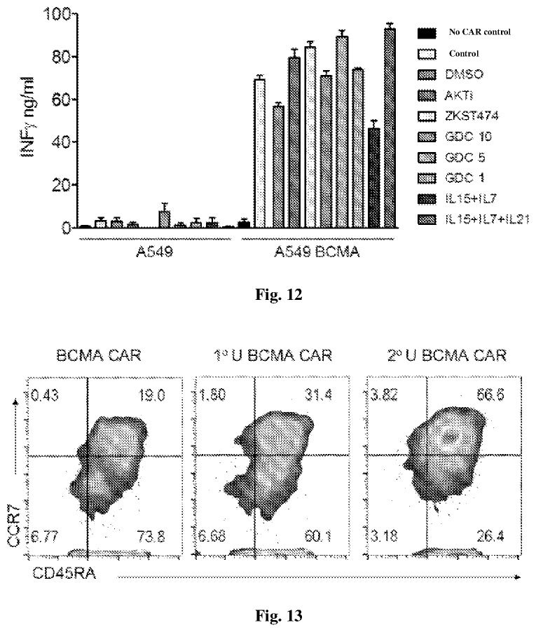

As shown in FIGS. 10-13. culturing T cells under different conditions, especially in the presence of different cytokines, adding IL1, IL7 and IL21 can significantly increase the enrichment of Tscm cells, promote cell growth, and improve the BCMA CAR transduction rate and increase the IFN-γ release.

All the literature mentioned herein is incorporated by reference in this application as if each literature is individually incorporated by reference. In addition, it should be understood that after reading the content of the present invention taught in the above, those skilled in the art can make various changes or modifications to the present invention, and these equivalent forms also fall within the scope defined by the accompanied claims of the present application.

1. A non-naturally occurring T cell population, characterized in that a proportion C1 of T memory stem cells (Tscm) in the T cell population is 50%, based on the total number of T cells in the T cell population.

2. The T cell population as described in claim 1, characterized in that the T memory stem cells comprise CAR-T cells.

3. The T cell population as described in claim 1, characterized in that the T memory stem cells comprise CCR⁷⁺CD45RA⁺T cells.

4. The T cell population as described in claim 1, characterized in that, in the T cell population, a proportion C2 of those expressing a biomarker CCR7 is 50%, preferably 60%, more preferably, 70%.

5. The T cell population as described in claim 1, characterized in that the CAR-T cells are universal CAR-T cells targeting tumor antigens.

6. A cell preparation, characterized in that, the cell preparation contains the non-naturally occurring T cell population described in claim 1 and a pharmaceutically acceptable carrier, diluent or excipient.

7. A method for preparing CAR-T cells, characterized in that, comprising steps of:
(a) providing an isolated T cell;
(b) amplifying and culturing the isolated T cells in the presence of IL15, IL7 and IL21, thereby obtaining cultured T cells; and
(c) transforming the cultured T cells to prepare CAR-T cells.

8. The method as described in claim 1, characterized in that, in step (a), the isolated T cells are selected from a group of: naive T cells (Tn cells), a Tn cell-enriched cell population, or total T cells, or a combination thereof.

9. The method as described in claim 1, characterized in that, in step (b), when the isolated T cells are amplified and cultured, the concentration of IL15 in the culture system is 1-200 ng/ml, preferably 3-100 ng/ml, more preferably 5-20 ng/ml; the concentration of IL7 is 0.5-50 ng/ml, preferably, 1-20 ng/ml, more preferably 3-10 ng/ml; and the concentration of IL21 is 1-100 ng/ml, preferably, 3-50 ng/ml, more preferably 5-20 ng/ml.

10. A method for preparing CAR-T cells, comprising steps of:
(i) providing an isolated Tn cell,
(ii) optionally, amplifying and culturing the isolated T cells in the presence of IL15, IL7 and IL21 to obtain cultured T cells; and
(iii) transforming the T cells to prepare CAR-T cells.
